(19) United States
(12) Patent Application Publication
(10) Pub. No.: US 2018/0267124 A1
(43) Pub. Date: Sep. 20, 2018

(54) MAGNETIC RESONANCE IMAGING APPARATUS AND MAGNETIC RESONANCE IMAGING METHOD

(71) Applicant: HITACHI, LTD., Tokyo (JP)

(72) Inventors: Yasuhiro KAMADA, Tokyo (JP); Yoshiyuki KUNUGI, Tokyo (JP)

(21) Appl. No.: 15/541,533

(22) PCT Filed: Jan. 19, 2016
(86) PCT No.: PCT/JP2016/051350

§ 371 Date: Jul. 5, 2017

(30) Foreign Application Priority Data
Feb. 6, 2015 (JP) 2015-022530

(51) Int. Cl. G01R 33/561; A61B 5/055; G01R 33/54; G01R 33/48; G01R 33/46

(52) U.S. Cl. CPC G01R 33/5611; A61B 5/055; G01R 33/4633; G01R 33/546; G01R 33/4822; G01R 33/543

(57) ABSTRACT

In an imaging method using k space low-frequency region data including a lot of useful information, in order to obtain an image with high quality without increasing measurement time by measuring a minimum required region in proper quantities, according to the present invention, pre-measurement is performed prior to main measurement, a rough shape of k space low-frequency region data is measured with respect to each signal reception channel, so as to be set as k space characteristics, and a range measured as a k space low-frequency region is specified in the measurement. The specified result is reflected in an imaging sequence, and thus k space low-frequency region data including useful information which can be used for a process is appropriately collected.

### TECHNICAL FIELD

The present invention relate to a magnetic resonance imaging technique, and particularly to an imaging technique using k space low-frequency region data.

### BACKGROUND ART

An MRI apparatus is an apparatus which measures an NMR signal (echo signal) generated by atomic nucleus spins forming an object, especially, tissues of a human body, and generates morphologies or functions of the head, the abdomen, and the limbs thereof as two-dimensional or three-dimensional images. In imaging, an NMR signal is added with different phase encodes and frequency encodes due to a gradient magnetic field. The measured NMR signal is subjected to two-dimensional or three-dimensional Fourier transform so as to be reconstructed as an image.

The measured NMR signal is disposed in a data space called a k space on a memory, and is thus called k space data. Data (so-called k space low-frequency region data) around the origin of the k space data has a greater signal value and includes more information (an object signal and a spatial distribution) than that in other regions. Thus, k space low-frequency region data is used in various types of imaging.

As an imaging method using this k space low-frequency region data, there is, for example, parallel imaging in which a k space is measured through thinning-out, and imaging is performed at a high speed (refer to NPL 1 and PTL 1). In the parallel imaging, a sensitivity distribution or a phase distribution of a reception channel, or the periodicity of k space data is obtained on the basis of k space low-frequency region data of each reception channel, and then an image is reconstructed by using an obtained result.

There is technique called compressed sensing in which repetitive calculation is performed on an image which is created on the basis of k space data measured through random thinning-out, and a complete image is restored (refer to PTL 2). In the compressed sensing technique, a low frequency region of the k space is measured with density higher than that of other regions in most cases.

Signal correction or the like is performed by using k space low-frequency region data according to a half estimation process of estimating non-measured data by using the conjugate symmetry of the k space or a process of combining pieces of data in a plurality of signal reception channels with each other by using complex numbers.

There is also a technique (PTL 3) in which a threshold value process is applied to measured k space low-frequency region data, and only data of a high signal is selectively used for a process, or a technique (NPL 2) in which only a meaningful signal component is extracted by applying singular value decomposition.

### CITATION LIST

### Patent Literature

PTL 1: Specification of U.S. Pat. No. 6,841,998

PTL 2: Specification of U.S. Pat. No. 7,646,924

PTL 3: JP-A-2013-42979

### Non-Patent Literature

NPL 1: Klass P. Pruessmann, Markus Weiger, Markus B. Scheidegger, and Peter Boesiger, “SENSE Sensitivity Encoding for Fast MRI”. Magnetic Resonance in Medicine 1999 42 p 952 to 962

NPL 2: P. Qu, J. Yuan, B. Wu, G. X. Shen, “Optimization of Regularization Parameter for GRAPPA Reconstruction”, Proc. Intl. Soc. Mag. Reson. Med. 2006 14 p 2474

### SUMMARY OF INVENTION

### Technical Problem

As mentioned above, a method of measuring k space low-frequency region data including a lot of useful information with high density is used for various processes. A signal intensity distribution (shape) of k space data in the k space, particularly, shape of k space low-frequency region data changes depending on imaging types such as an FOV or a section, a sequence kind, and image contrast. A position (peak position) of the highest signal is shifted depending on a phase distribution of a reception channel. Thus, a peak position of k space data cannot be said to be located at the origin of the k space, and is shifted to different positions for each signal reception channel.

The k space low-frequency region data has the above-described property, and there is a variation in a shape thereof. Thus, in order to reliably measure k space low-frequency region data, it is necessary to acquire excessive data by regarding a region considerably separated from the origin of the k space as a low frequency region of the k space.

Since an excessively wide region is regarded as a k space low frequency region, and k space low-frequency region data is acquired, data which is not inherently k space low-frequency region data is used for a process. Consequently, artifacts are generated, or measurement time increases.

The present invention has been made in consideration of the circumstances, and an object thereof is to obtain an image with high quality without increasing measurement time by measuring a minimum required region in proper quantities in an imaging method using k space low-frequency region data including a lot of useful information.

### Solution to Problem

According to the present invention, pre-measurement is performed prior to main measurement, a rough shape of k space low-frequency region data is measured with respect to each signal reception channel, so as to be set as k space characteristics, and a range measured as a k space low-frequency region is specified in the measurement. The specified result is reflected in an imaging sequence, and thus k space low-frequency region data including useful information which can be used for a process is appropriately collected.

### Advantageous Effects of Invention

It is possible to obtain an image with high quality without increasing measurement time by measuring a minimum required region in proper quantities in an imaging method using k space low-frequency region data including a lot of useful information.

### BRIEF DESCRIPTION OF DRAWINGS

FIG. 1 is a block diagram illustrating the entire configuration of an MRI apparatus of a first embodiment.

FIG. 2 is a functional block diagram of a control system of the first, embodiment.

FIGS. 3(a) and 3(b) are diagrams for explaining a method of determining a k space reference position in the first embodiment.

FIGS. 4(a) to 4(c) are diagrams for explaining a method of determining a k space low-frequency region measurement width in the first embodiment.

FIGS. 5(a) to 5(d) are diagrams for explaining specific examples of imaging sequence adjustment in the first embodiment.

FIGS. 6(a) to 6(d) are diagrams for explaining specific examples of imaging sequence adjustment in the first embodiment.

FIG. 7 is a flowchart illustrating a k space characteristic information determination process and an imaging sequence adjustment process in the first embodiment.

FIGS. 8(a) and 8(b) are diagrams for explaining a method of determining a k space reference position in a modification example of the first embodiment.

FIGS. 9(a) to 9(c) axe diagrams for explaining a method of determining a k space low-frequency region measurement width in a modification example of the first embodiment.

FIG. 10 is a functional block diagram of a control system of a second embodiment.

FIGS. 11(a) to 11(c) are diagrams for explaining an instruction reception screen of the second embodiment.

FIG. 12(a) is a diagram for explaining a k space characteristic information estimation process in a third embodiment, and FIG. 12(b) is a diagram for explaining a k space characteristic information estimation process in a modification example of the third embodiment.

FIG. 13 is a flowchart illustrating a k space characteristic information determination process and an imaging sequence adjustment process in the third embodiment.

FIG. 14 is a functional block diagram of a control system of a fourth embodiment.

FIG. 15 is a flowchart illustrating a k space characteristic information determination process, an imaging sequence adjustment process, and a reception gain setting process in the fourth embodiment.

### DESCRIPTION OF EMBODIMENTS

### First Embodiment

Hereinafter, with reference to the drawings, a description will be made of a first embodiment to which the present invention is applied. Throughout the drawings for explaining the respective embodiments, among constituent elements given the same names and the same reference numerals, constituent elements having the same functions will not be described repeatedly.

### MRI Apparatus Configuration

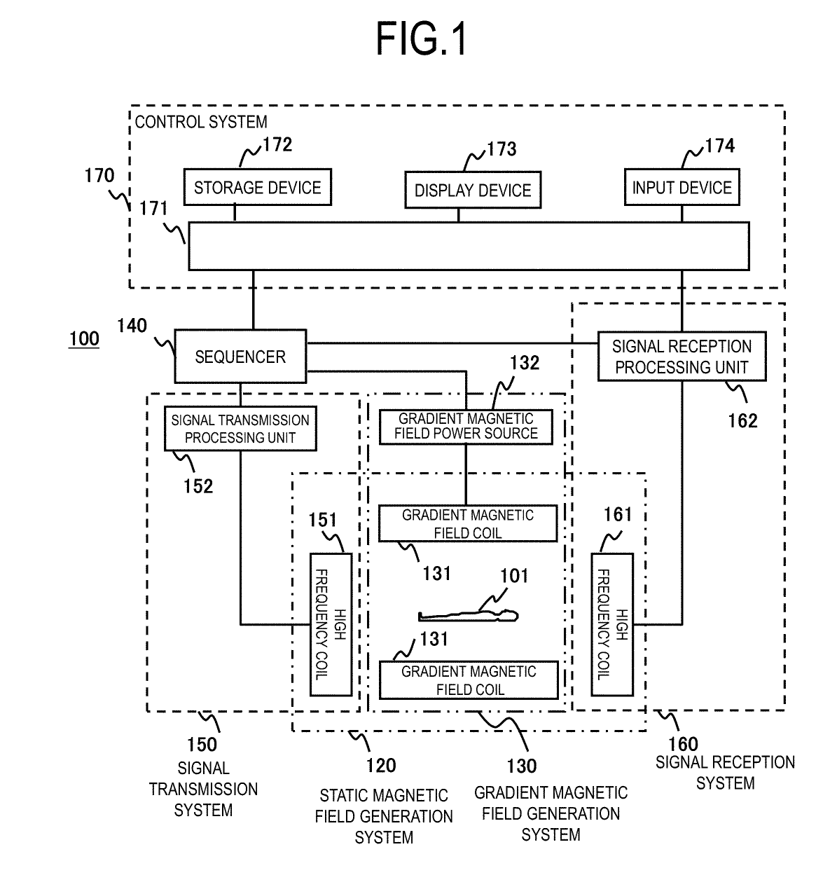

First, the entire summary of an example of an MRI apparatus of the present embodiment will be described. FIG. 1 is a block diagram illustrating the entire configuration of an MRI apparatus 100 of the present embodiment. The MRI apparatus 100 of the present embodiment obtains a tomographic image of an object by using an NMR phenomenon, and, as illustrated in FIG. 1, includes a static magnetic field generation system 120, a gradient magnetic field generation system 130, a signal transmission system 150, a signal reception system 160, a control system 170, and a sequencer 140.

The static magnetic field generation system 120 generates a uniform static magnetic field in a space around an object 101 in a direction orthogonal to a body axis of the object in a vertical magnetic field type, and generates a uniform static magnetic field in a body axis direction in a horizontal magnetic field type, and includes a permanent magnet type, normal conducting type, or superconducting type static magnetic field generation source disposed around the object 101.

The gradient magnetic field generation system 130 includes gradient magnetic field coils 131 which are wound in three-axis directions of X, Y, and Z in a coordinate system (apparatus coordinate system) of the MRI apparatus 100, and a gradient magnetic field power source 132 which drives the gradient magnetic field coils, and applies gradient magnetic fields Gx, Gy and Gz in the three-axis directions of X, Y, and Z by driving the gradient magnetic field power source 132 for each of the gradient magnetic field coils 131 in response to a command from the sequencer 140.

The signal transmission system 150 irradiates the object 101 with a high frequency magnetic field pulse (hereinafter, referred to as an “RF pulse”) in order to cause nuclear magnetic resonance in an atomic nucleus spin of an atom forming a living body tissue of the object 101, and includes a signal transmission processing unit 152 provided with a high frequency oscillator (synthesizer), a modulator, and a high frequency amplifier, and a high frequency coil (signal transmission coil) 151 on the signal transmission side. The high frequency oscillator generates an RF pulse, and outputs the RF pulse at a timing based on a command from the sequencer 140.

The modulator amplitude-modulates the output RF pulse, and the high frequency amplifier amplifies the amplitude-modulated RF pulse, and supplies the RF pulse to the signal transmission coil 151 disposed near the object 101. The signal transmission coil 151 irradiates the object 101 with the supplied RF pulse.

The signal reception system 160 detects a nuclear magnetic resonance signal (an echo signal or an NMR signal) emitted due to nuclear magnetic resonance of atomic nucleus spins forming a living body tissue of the object 101, and includes a high frequency coil (signal reception coil) 161 on the signal reception side, and a signal reception processing unit 162 provided with a combiner, an amplifier, a quadrature phase detector, and an A/D converter.

The signal reception coil 161 which is a multi-channel coil having a plurality of signal reception channels is disposed near the object 101, and detects an NMR signal (received signal) which is caused by the object 101 in response to electromagnetic waves applied front the signal transmission coil 151, in each channel. The received signal in each channel which is amplified by the signal reception processing unit 162 is detected at a timing based on a command from the sequencer 140 so as to be converted into a digital signal, and is then transmitted to the control system 170 for each channel as k space data.

The sequencer 140 repeatedly applies an RF pulse and a gradient magnetic field pulse according to a predetermined pulse sequence. The pulse sequence describes timings or intensities of a high frequency magnetic field, a gradient magnetic field, and signal reception, and is held in the control system 170 in advance. The sequencer 140 is operated in response to an instruction from the control system 170, and transmits various commands which are required to collect tomographic image data of the object 101, to the signal transmission system 150, the gradient magnetic field generation system 130, and the signal reception system 160.

The control system 170 performs control of the entire operation of the MRI apparatus 100, signal processing, various types of calculation such as image reconstruction, and display and preservation of a process result, and includes a CPU 171, a storage device 172, a display device 173, and an input device 174. The storage device 172 is formed of an internal storage device such as a hard disk, and an external storage device such as an externally attached hard disk, an optical disc, and a magnetic disk. The display device 173 is a display device such as a CRT or a liquid crystal display.

The input device 174 is an interface for inputting various pieces of control information regarding the MRI apparatus 100 or control information regarding processes performed by the control system 170, and includes, for example, a track ball or a mouse and a keyboard. The input device 174 is disposed near the display device 173. An operator inputs instructions and data which are required for various processes in the MRI apparatus 100 in an interactive manner via the input device 174 while viewing the display device 173.

The CPU 171 performs a program held in advance in the storage device 172 in response to an instruction which is input by the operator, so as to realize control of an operation of the MRI apparatus 100, and respective processes such as data processing in the control system 170, and functions thereof. For example, if data from the signal reception system 160 is input to the control system 170, the CPU 171 performs processes such as signal process and image reconstruction, displays a tomographic image of the object 101 as a result thereof on the display device 173, and stores the tomographic image in the storage device 172.

Some or all of the functions of the control system 170 may be realized by hardware such as an application specific integrated circuit (ASIC) or a field-programmable gate array (FPGA). Various pieces of data used for processing each function, and various pieces of data generated during processing are stored in the storage device 172.

The signal transmission coil 151 and the gradient magnetic field coils 131 are provided to oppose the object 101 in a vertical magnetic field type, and to surround the object 101 in a horizontal magnetic field type, in a static magnetic field space of the static magnetic field generation system 120 into which the object 101 is inserted. The signal reception coil 161 is provided to oppose or surround the object 101.

Currently, an imaging target nuclide of the MRI apparatus, which is clinically popular, is a hydrogen nucleus (proton) which is a main constituent substance of the object 101. In the MRI apparatus 100, information regarding a spatial distribution of the proton density and information regarding a spatial distribution of the relaxation time of an excitation state is generated as an image so that a morphology or a function of the human head, abdomen, limbs, or the like is imaged in a two-dimensional or three-dimensional manner.

### Functional Configuration of Control System

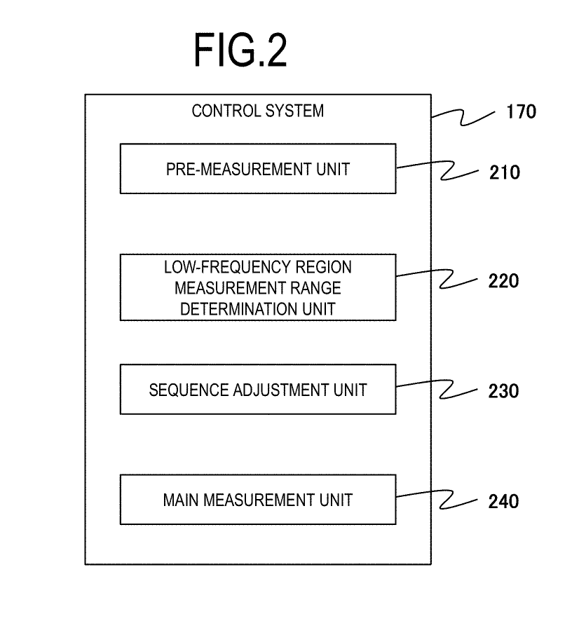

The control system 170 of the present invention includes, as illustrated in FIG. 2, a pre-measurement unit 210 which measures k space data in a k space low-frequency region which is a predefined low-frequency region range of the k space by using the same imaging sequence as that in main measurement performed for acquiring an image; a low-frequency region measurement range determination unit 220 which obtains k space characteristic information for specifying a k space low-frequency region data measurement range in which k space low-frequency region data can be measured by using k space data collected by the pre-measurement unit 210; a sequence adjustment unit 230 which adjusts an imaging sequence so that k space low-frequency region data is measured in a k space low-frequency region data measurement range; and a main measurement unit 240 which performs main measurement by using an imaging sequence adjusted by the sequence adjustment unit.

In the present embodiment, a predetermined region around the origin of the k space will be referred to as a k space low-frequency region. As described above, data which has a greater signal value and includes more information than that in other regions will be referred to as k space low-frequency region data.

### Pre-Measurement Unit

The pre-measurement unit 210 measures a predefined search range at a predefined pitch (interval) by using the same sequence as a sequence used for main measurement.

The search range is set by a user. Alternatively, the search range is predefined. In this case, the search range is set to reliably include a k space low-frequency region including useful information. For example, the search range is set to be ±16 encodes in terms of phase encode (including slice encode in three-dimensional measurement) amount. The search range may be changed depending on parameters (for example, a pulse sequence type, a phase encode/slice encode direction, the multiple number of times of speed in parallel imaging, and a compression ratio in compressed sensing) for main measurement.

A search pitch (interval) may be the same as that in main measurement, but may be set to be twice or more the main measurement pitch in order to reduce search time.

The pre-measurement unit 210 measures an echo signal at each point in the set search range. The signal reception processing unit 162 performs a reception process on the obtained echo signal so as to obtain k space data, obtains an absolute value of the k space data, and preserves the absolute value of the k space data in a memory (k space). The k space data is preserved in a k space memory prepared for each channel. Hereinafter, a distribution of the k space data for each channel, obtained through measurement in the pre-measurement unit 210 will be referred to as k space search data.

### Low-Frequency Region Measurement Range Determination Unit 220

The low-frequency region measurement range determination unit 220 determines a substantial k space range in which k space low-frequency region data can be measured by using k space search data. Hereinafter, a substantial k space range in which k space low-frequency region data can be measured will be referred to as a k space low-frequency region data measurement range. In the present embodiment, the low-frequency region measurement range determination unit 220 calculates a signal intensity distribution (a shape of k space low-frequency region data) of k space data around a k space low-frequency region as k space characteristic information by using the k space data. The k space low-frequency region data measurement range is defined by calculated k space characteristics.

The k space characteristic information includes a k space reference position which is a position where the signal intensity of k space data is the maximum, and a k space low-frequency region measurement width which is defined according to the signal intensity of the k space data. In the present embodiment, a k space reference position is determined, and then a k space low-frequency region measurement width is determined.

First, the low-frequency region measurement range determination unit 220 determines a k space reference position.

Here, the k space reference position is defined as the substantial k space origin in consideration of a peak shift caused by geometry such as an FOV or a section, a peak shift caused by an imaging sequence, and a peak shift caused by a phase distribution of each signal reception channel. The k space reference position is specified by a phase encode amount.

The low-frequency region measurement range determination unit 220 combines pieces of k space data received in the respective channels of the signal reception coil 161 with each other, so as to obtain combined data, and sets a position where the signal intensity of the combined data is the maximum as the k space reference position.

The low-frequency region measurement range determination unit 220 combines pieces of k space search data received in the respective channels with each other so as to measure a shape as a result of the combination, and sets a position (peak position) where the signal intensity of the combined data is the maximum as the k space reference position. In other words, a phase encode amount ky indicating a peak is determined as the k space reference position.

A k space shape in each channel may be calculated on the basis of k space search data in each channel, and calculated k space shapes in the respective channels may be combined with each other so that the k space reference position is determined.

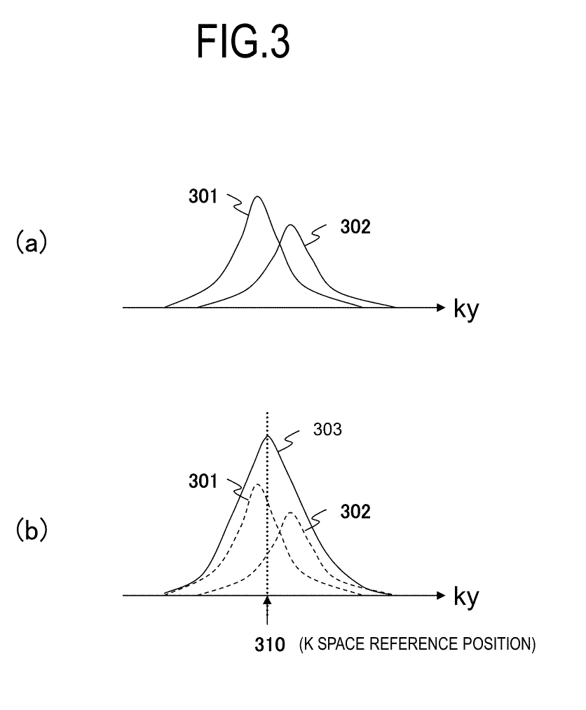

Hereinafter, details thereof will be described with reference to FIGS. 3(a) and 3(b). Here, the number of channels is assumed to be two. FIG. 3(a) displays shapes 301 and 302 of k space search data in the respective channels (channel 1 and channel 2) in a phase encode direction. Here, since a frequency encode direction (kx direction) is not a processing target, data is illustrated in which the maximum value of k space search data is projected in the kx direction.

FIG. 3(b) illustrates a shape 303 of addition k space search data obtained by adding (combining) the shape 301 of the k space search data in the channel 1 to the shape 302 of the k space search data in the channel 2.

The low-frequency region measurement range determination unit 220 sets a phase encode amount in which the shape 303 of the addition k space search data is the maximum, as a k space reference position 310.

Next, the low-frequency region measurement range determination unit 220 specifies a k space low-frequency region measurement width.

Here, the k space low-frequency region measurement width is defined as a k space signal range which may be substantially called a k space low-frequency region in consideration of a k space change caused by geometry such as an FOV or a section, a k space change caused by imaging sequence, and a k space change caused by a phase distribution of each reception channel.

The k space low-frequency region measurement width is specified by the number of phase encode steps.

The low-frequency region measurement range determination unit 220 sets a range which includes the k space reference position 310 and in which combined data obtained by combining pieces of k space data received in the respective channels of the signal reception coil 161 with each other satisfies a predefined condition, as the k space low-frequency region measurement width. For example, a width (the number of phase encode steps) of combined data having a signal value of a predefined threshold value or greater is specified as the k space low-frequency region measurement width in the shape 303 of the addition k space search data.

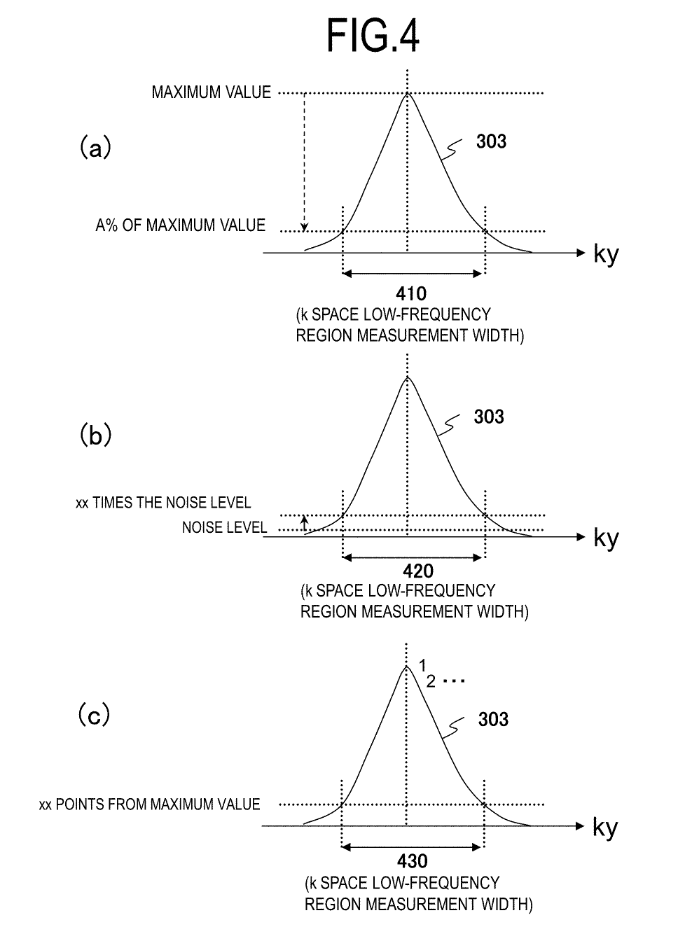

Details thereof will be described with reference to FIGS. 4(a) to 4(c). FIGS. 4(a) and 4(b) illustrate k space low-frequency region measurement widths in a case where a predefined condition is that a signal value (signal intensity) is equal to or greater than a predetermined threshold value. FIG. 4(a) illustrates a k space low-frequency region measurement width 410 in a case where a threshold value is defined to be A% (where A is a real number which is more than 0 and is less than 100) of the maximum value, and FIG. 4(b) illustrates a k space low-frequency region measurement width 420 in a case where a threshold value is defined to be B times (where B is a real number more than 1) the noise level.

Instead of setting a condition by using a threshold value, for example, as illustrated in FIG. 4(c), the condition may be set by using the number of data points. In other words, a predefined condition may be set by using a predetermined number (XX) of pieces of data having a great signal value. Specifically, data is counted in descending order of a signal value with the shape 303 of the addition k space search data, and a region until reaching a predefined number of points (XX points) is set as the k space low-frequency region measurement width 430. Points of a predefined number (phase encode steps of the number) with a phase encode position having the maximum value as the center may be set as the k space low-frequency region measurement width.

The low-frequency region measurement range determination unit 220 outputs a value of the phase encode amount (ky) as the k space reference position, and outputs the number of phase encode steps as the k space low-frequency region measurement width. The k space low-frequency region data measurement range is a range of the k space low-frequency region measurement width with the k space reference position as the center.

However, if the k space low-frequency region data measurement range is specified in the above-described way, a hole or an isolated point may be generated in a measurement region. Thus, the hole or the isolated point may be removed by applying a general expansion or contraction process on a defined region.

### Sequence Adjustment Unit

The sequence adjustment unit 230 adjusts an imaging sequence so that a k space low-frequency region data measurement range defined by k space characteristic information determined by the low-frequency region measurement range determination unit 220 is measured. In other words, when k space low-frequency region data is acquired, an imaging sequence is adjusted so that data regarding a k space low-frequency region measurement width is acquired with a k space reference position as the center of a k space low-frequency region.

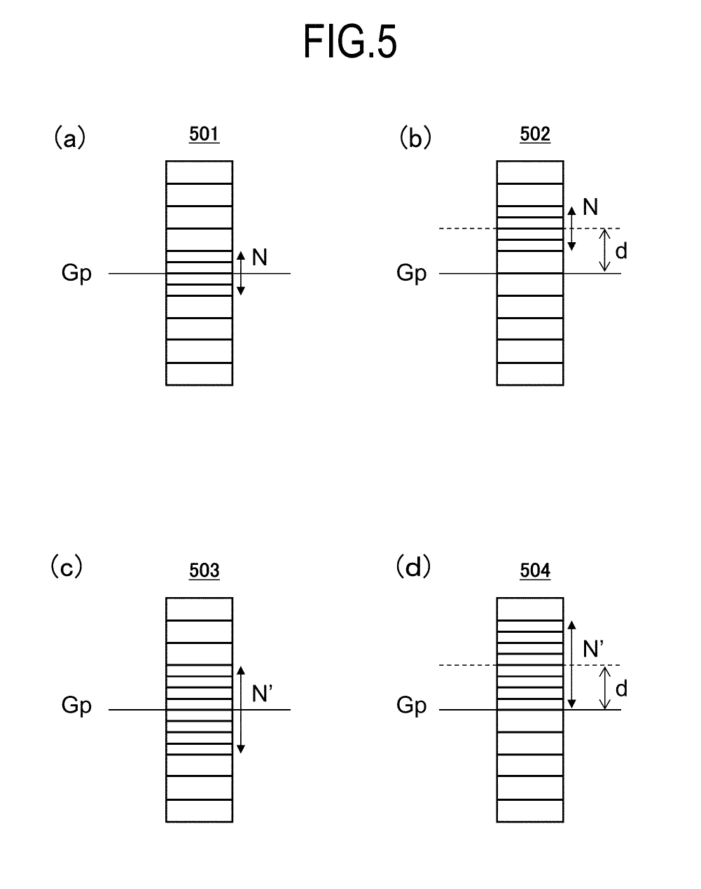

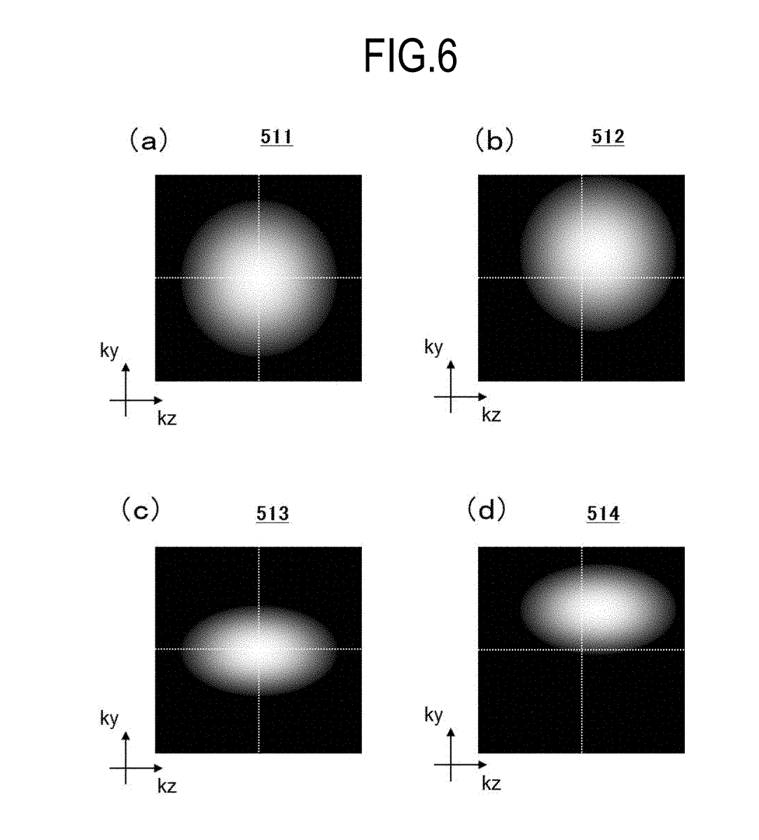

Specific examples are illustrated in FIGS. 5(a) to 5(d) and FIGS. 6(a) to 6(d).

With reference to FIGS. 5(a) to 5(d), a description will be made of adjustment of a phase encode amount Gp in a case of parallel imaging in which a k space low-frequency region is densely acquired in a phase encode (Gp) direction. Each figure illustrates a phase encode gradient magnetic field intensity Gp.

In the parallel imaging, a k space low-frequency region is densely acquired. In the related art, as illustrated in FIG. 5(a), a range of a predetermined number of encode steps N (where N is an integer of 1 or more) is densely acquired with the k space origin as the center. As illustrated in FIG. 5(a), a k space reference position is set to be a phase encode amount of 0 (ky=0), the number of phase encode steps of a k space low-frequency region width is indicated by N, and an initial state of the phase encode gradient magnetic field Gp in an imaging sequence is 501.

FIG. 5(b) illustrates a phase encode gradient magnetic field 502 in a case where the low-frequency region measurement range determination unit 220 determines a k space reference position as d (where ky=d, and d≠0), and a k space low-frequency region measurement width is determined as N. The sequence adjustment unit 230 adjusts the imaging sequence so that the phase encode gradient magnetic field Gp in the imaging sequence becomes the phase encode gradient magnetic field 502.

FIG. 5(c) illustrates a phase encode gradient magnetic field 503 in a case where the low-frequency region measurement range determination unit 220 determines a k space reference position as 0 (where ky=0), and a k space low-frequency region measurement width is determined as N′ (where N is an integer of 1 or more satisfying (N≠N′). The sequence adjustment unit 230 adjusts the imaging sequence so that the phase encode gradient magnetic field Gp in the imaging sequence becomes the phase encode gradient magnetic field 503.

FIG. 5(d) illustrates a phase encode gradient magnetic field 504 in a case where the low-frequency region measurement range determination unit 220 determines a k space reference position as d (where ky=d), and a k space low-frequency region width is determined as N′. The sequence adjustment unit 230 adjusts the imaging sequence so that the phase encode gradient magnetic field Gp in the imaging sequence becomes the phase encode gradient magnetic field 504.

With reference to FIGS. 6(a) to 6(d), a description will be made of adjustment of a sampling density in a case of measurement (for example, compressed sensing) in which the sampling density varies in a seamless manner from a k space low-frequency region to a k space high-frequency region. Each figure illustrates a sampling density for sampling according to an imaging sequence in a ky-kz space.

FIG. 6(a) illustrates a sampling density 511 in an imaging sequence in an initial state. A width of a k space low-frequency region is indicated by N. In the initial state, the sampling density 511 is defined by a function obtained by combining a predetermined function with the k space origin as the center, for example, a two-dimensional function (ky, kz) as expressed in the following Equation (1) with a normal distribution.

$$\begin{matrix}Density ( ky , kz ) = {M}_{0} * \frac{1}{\sqrt{2 {πσ}_{ky}^{2}}} exp ( - \frac{{( ky - {μ}_{ky} )}^{2}}{2 {σ}_{ky}^{2}} ) * \frac{1}{\sqrt{2 {πσ}_{kz}^{2}}} exp ( - \frac{{( kz - {μ}_{kz} )}^{2}}{2 {σ}_{kz}^{2}} ) \\ = {M}_{0} * \frac{1}{2 {πσ}_{ky}^{2} {σ}_{kz}^{2}} exp ( \frac{{( ky - {σ}_{ky} )}^{2}}{2 {σ}_{ky}^{2}} - \frac{{( kz - {μ}_{kz} )}^{2}}{2 {σ}_{kz}^{2}} )\end{matrix} \qquad ( 1 )$$

Here, μ_{ky} and μ_{kz} respectively indicate central coordinates in the ky direction and the kz direction, σ_{ky}² and σ_{kz}² respectively indicate variances in the ky direction and the kz direction, and M₀ indicates an adjustment coefficient. These values are appropriately set, and thus it is possible to realize sampling in which a density varies from a k space low-frequency region to a k space high-frequency region. In FIG. 6(a), μ_{ky}=0 and μ_{kz}=0, and σ_{ky}²=N/2 and σ_{kz}²=N/2 are defined.

FIG. 6(b) illustrates a sampling density 512 in a case where the low-frequency region measurement range determination unit 220 determines a k space reference position as D (where ky=d1, kz=d2, d1≠0, and d2≠0) and a k space low-frequency region measurement width as N, and the sequence adjustment unit 230 adjusts an imaging sequence according thereto. In this case, the sampling density 512 is defined as μ_{ky}=d1 and μ_{kz}=d2, and σ_{ky}²=N/2 and σ_{kz}²=N/2 by using the same function as in the initial state.

FIG. 6(c) illustrates a sampling density 513 in a case where the low-frequency region measurement range determination unit 220 determines a k space reference position as 0 and a k space low-frequency region measurement width as N1 in the ky direction and N2 (N1 and N2 are integers of 1 or more respectively satisfying N1≠N and N2≠N) in the kz direction, and the sequence adjustment unit 230 adjusts an imaging sequence according thereto. In this case, the sampling density 513 is defined by a function modified as in μ_{ky}=0 and μ_{kz}=0, and σ_{ky}²=N1/2 and σ_{kz}²=N2/2 by using the same function as in the initial state.

FIG. 6(d) illustrates a sampling density 514 in a case where the low-frequency region measurement range determination unit 220 determines a k space reference position as D and a k space low-frequency region measurement width as N1 in the ky direction and N2 in the kz direction, and the sequence adjustment unit 230 adjusts an imaging sequence according thereto. In this case, the sampling density 514 is defined by a function modified as in μ_{ky}=d1 and μ_{kz}=d2, and σ_{ky}²=N1/2 and σ_{kz}²=N2/2 by using the same function as in the initial state.

According to the present embodiment, since a k space reference position and a k space low-frequency region measurement width are specified, for example, in a case where k space low-frequency region measurement widths in the ky direction and the kz direction are different from each other as in the sampling density 513, measurement may be performed by modifying a function. A function in which both of a k space reference position and a k space low-frequency region measurement width are modified may be used as in the sampling density 514.

### Main Measurement Unit

The main measurement unit 240 performs measurement by using an imaging sequence adjusted by the sequence adjustment unit 230, and obtains an image.

### k Space Characteristic Information Determination and Imaging Sequence Adjustment Processes

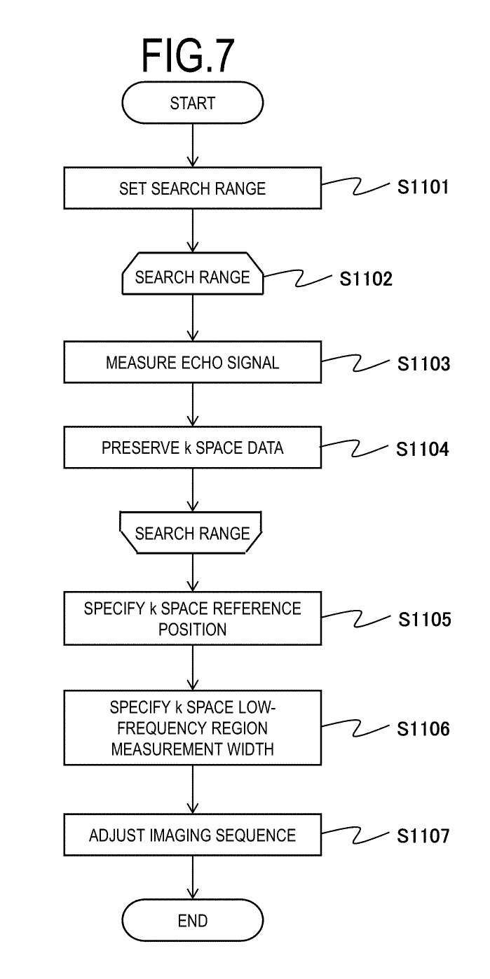

A description will toe made of a flow of k space characteristic information determination and imaging sequence adjustment processes in the present embodiment. FIG. 7 illustrates a processing flow of the present process. The present process is performed right after an instruction for starting each scanning is given prior to main measurement.

The pre-measurement unit 210 sets a search range for pre-measurement in order to determine k space characteristic information (step S1101), and performs pre-measurement (steps S1102 to S1104). Here, an echo signal is measured in the predefined search range, and a k space signal value (k space data) for determining a range is preserved.

If k space data is measured in all search ranges, the low-frequency region measurement range determination unit 220 determines a k space reference position among pieces of the k space characteristic information toy using the measured k space search data (step S1105). Thereafter, the low-frequency region measurement range determination unit 220 determines a k space low-frequency region measurement width among pieces of the k space characteristic information (step S1106).

The sequence adjustment unit 230 adjusts an imaging sequence on the basis of the k space reference position and the k space low-frequency region measurement width (step S1107).

As described above, the MRI apparatus of the present embodiment includes the pre-measurement unit 210 which measures k space data in a predefined range in a k space low-frequency region by using the same imaging sequence as that of main measurement performed for acquiring an image; the low-frequency region measurement range determination unit 220 which obtains k space characteristic information for specifying a k space low-frequency region data measurement range in which k space low-frequency region data can be measured by using k space data collected by the pre-measurement unit 210; the sequence adjustment unit 230 which adjusts an imaging sequence so that k space data in the k space low-frequency region data measurement range is measured as the k space low-frequency region data; and the main measurement unit 240 which performs the main measurement by using an imaging sequence adjusted by the sequence adjustment unit 230.

In this case, the k space characteristic information includes a k space reference position which is a position where the signal intensity of the k space data is the maximum, and the sequence adjustment unit 230 adjusts the imaging sequence so that the k space low-frequency region data is measured from a range centering on the k space reference position.

The k space characteristic information includes a k space low-frequency region measurement width defined according to the signal intensity of the k space data, and the sequence adjustment unit 230 adjusts the imaging sequence so that the k space low-frequency region data is measured from a range of the k space low-frequency region measurement width.

As mentioned above, according to the present embodiment, a substantial k space low-frequency region data measurement range is determined in which k space low-frequency region data having a greater signal value and including more information than that in other regions can be measured by using a result of pre-measuring the vicinity of the predefined k space origin. Main measurement is performed according to a sequence in which the k space low-frequency region data measurement range is reflected.

Therefore, according to the present embodiment, even in a case where any FOV or section, sequence type, image contrast, and signal reception coil are used, an actual k space data shape can be specified, and thus it is possible to efficiently collect k space low-frequency region data appropriately (without excessive collecting or deficient collecting).

### Modification Examples of k Space Characteristic Determination Method

In the above-described embodiment, the low-frequency region measurement range determination unit 220 determines a k space reference position and a k space low-frequency region measurement width as k space characteristic information, but may not necessarily determine both of the two. Either one thereof may be determined.

In the above-described embodiment, a k space reference position is determined on the basis of a shape of a result of combining pieces of k space search data received in the respective channels with each other. However, this method is only an example. For example, a position where the signal intensity is the maximum may be specified with respect to each piece of k space data (k space search data) received in the respective channels of the signal reception coil 161, and a centroid position of each specified result may be set as a k space reference position.

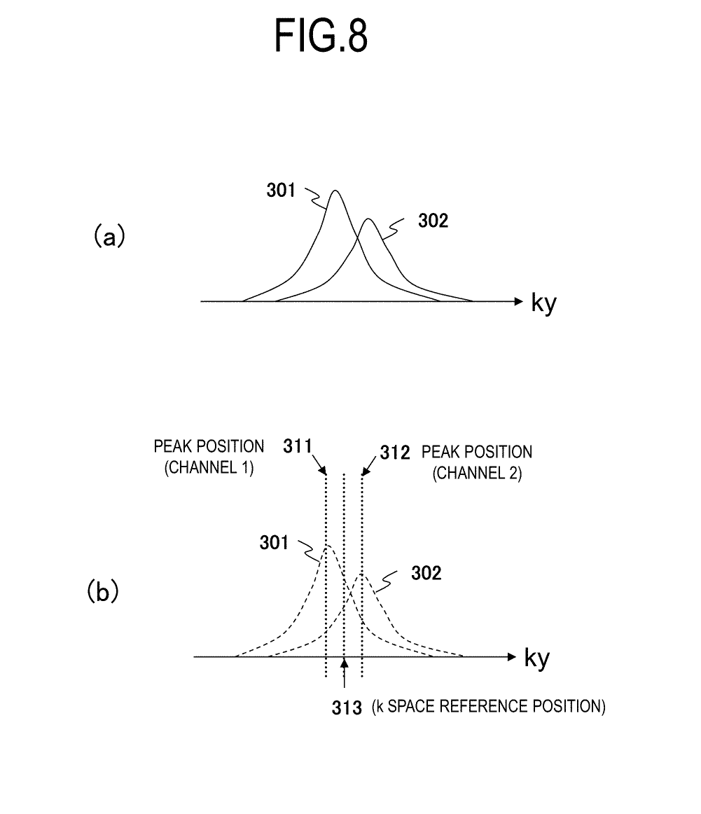

This method will be described with reference to FIGS. 8(a) and 8(b). Here, a description will be made of a case of two channels in the same manner as described as an example.

First, the low-frequency region measurement range determination unit 220 measures shapes 301 and 302 of k space search data in the respective channels (channel 1 and channel 2). A peak position 311 of the channel 1 and a peak position 312 of the channel 2 are determined. Finally, a centroid position (an average value of a phase encode amount) of both of the peak positions 311 and 312 is determined as a k space reference position 313.

The peak positions 311 and 312 in the respective channels may be weighted with the peak intensity and are averaged, and a result position may be determined as a k space reference position. A midpoint between the maximum and the minimum of the peak positions 311 and 312 in the respective channels may be obtained so as to be determined as a k space reference position.

As mentioned above, since a k space reference position is determined by using pieces of k space search data in respective channels, it is possible to obtain a substantial k space origin position in consideration of not only a peak shift caused by geometry such as an FOV or a section and a peak shift caused by an imaging sequence but also a peak shift caused by a phase distribution of each signal reception channel.

In this case, regarding the k space low-frequency region measurement width, for example, a region is specified which includes the k space reference position 313 and in which k space data received in each channel of the signal reception coil 161 satisfies a predefined condition, and respective specified results are combined with each other so as to be set as the k space low-frequency region measurement width. The predefined condition is, for example, that a signal value is equal to or greater than a predefined threshold value, or a predetermined number in descending order of a signal value. The combination may be any one of AND combination, OR combination, and centroid combination. However, in this case, the k space low-frequency region measurement width is preferably defined to include the specified regions for the signal reception channels.

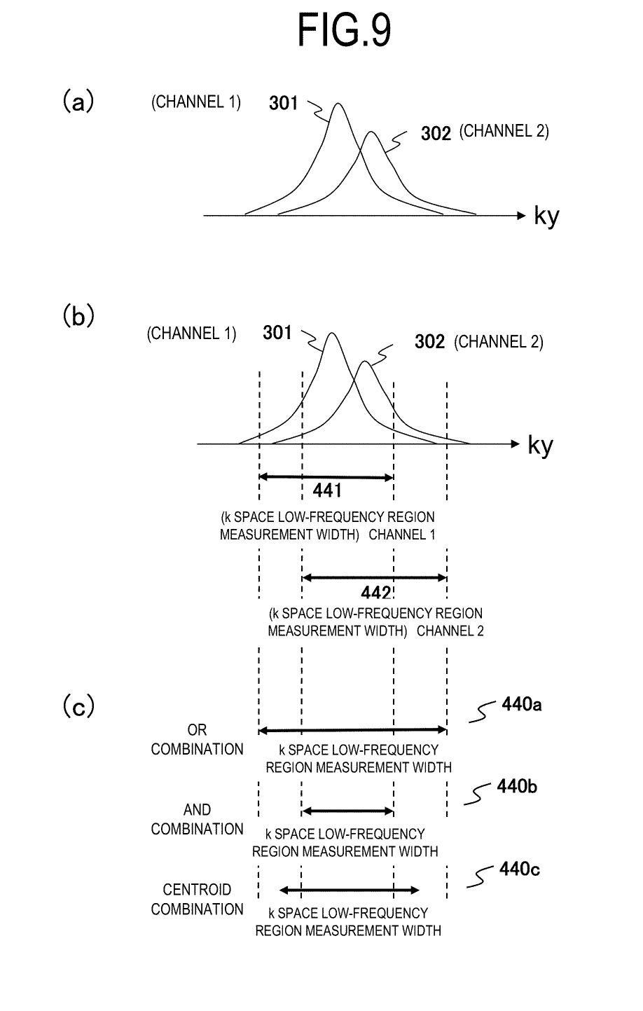

Details of the method will be described with reference to FIGS. 9(a) to 9(c). Here, a description will be made of a case of two channels in the same manner as described as an example.

FIG. 9(a) illustrates that shapes 301 and 302 of k space search data in the respective channels (channel 1 and channel 2) in a phase encode direction (ky direction). The low-frequency region measurement range determination unit 220 calculates ranges (k space low-frequency region measurement widths) 441 and 442 of a predetermined threshold value or greater with respect to the shapes 301 and 302 of the k space search data in the respective channels. A calculation method is the same as the method of calculating of a k space low-frequency region measurement width of the shape 303 of the addition k space search data in the above-described embodiment.

The calculated k space low-frequency region measurement widths 441 and 442 in the respective channels are combined with each other according to OR combination (440a), AND combination (440b), or centroid combination (440c), and a result, thereof is used as a k space signal range in the measurement.

Details of the OR combination, the AND combination, and the centroid combination are respectively expressed by the following Equations (2-1) to (4-2). Here, M indicates the number of channels, m indicates a channel number (1 to M), ky_s(m) indicates a start point coordinate of a k space low-frequency region measurement width in a channel m, ky_s(m) indicates an end point coordinate of the k space low-frequency region measurement width in the channel m, ky_s indicates a start point coordinate of a combined k space low-frequency region measurement width, ky_e indicates an end point coordinate of a combined k space low-frequency region measurement width, min( ) indicates an operator for obtaining the minimum value in an array, max( ) indicates an operator for obtaining the maximum value in the array, and mean( ) indicates an operator for obtaining a mean value in the array.

OR Combination

ky_s=min(ky_s(1), ky_s(2), . . . ) (2-1)

ky_e=max(ky_e(1), ky_e(2), . . . ) (2-2)

AND Combination

ky_s=max(ky_s(1), ky_s(2), . . . ) (3-1)

ky_e=min(ky_e(1), ky_e(2), . . . ) (3-2)

Centroid Combination

ky_s=mean(ky_s(1), ky_s(2), . . . ) (4-1)

ky_e=mean(ky_e(1), ky_e(2), . . . ) (4-2)

In addition, k space characteristic information (a k space reference position and a k space low-frequency region measurement width) in each signal reception channel, calculated according to the method of this modification example, may be used without being changed depending on a processing aspect. For example, this case is a case (half estimation, compressing sensing, or the like) where a process is performed by using k space low-frequency region data independently for each signal reception channel.

On the other hand, in a case (parallel imaging, channel complex combination, or the like) of a process using a correlation between signal reception channels, in order to accurately estimate a relationship between signal reception channels, instead of extracting data from k space characteristic information for each channel, k space characteristic information may be calculated according to the method of the above-described embodiment by using measured k space low-frequency region data as it is.

In the present, embodiment and the modification example, k space characteristic information (a k space reference position and a k space low-frequency region measurement width) is determined by using k space search data in which the maximum value is projected in a kx direction.

However, k space characteristic information may be determined on a two-dimensional plane of kx-ky instead of projection in the kx direction. A determination method may be the same as described above.

In a case where there are a plurality of pieces of k space search data due to factors other than signal reception channels, such as multi-slice measurement or multi-echo measurement, k space characteristic information may be determined for each slice or each echo. However, there is a case where it is difficult to switch between measured coordinates of the k space for each slice or for each echo from the viewpoint of a pulse sequence shape. In this case, pieces of k space search data may foe added together in multi-slice directions or multi-echo directions, so as to be treated as a single piece of data.

### Second Embodiment

A second embodiment of the present invention will be described. In the present embodiment, an instruction for an adjustment result is received from a user.

The number of acquired echoes may change depending on a k space low-frequency region data measurement range specified on the basis of k space search data, and thus measurement time may change (increase or decrease) from expected time. In the present embodiment, particularly, in a case where measurement time increases, a user is allowed to select whether measurement is continuously performed by permitting an increase in measurement time, or measurement time is maintained by changing measurement parameters (for example, a resolution (the number of measurement matrices), and TR).

In the following description, an imaging sequence which is generated according to initially set imaging conditions will be referred to as an initial sequence, and an imaging sequence adjusted by the sequence adjustment unit 230 will be referred to as an adjusted sequence.

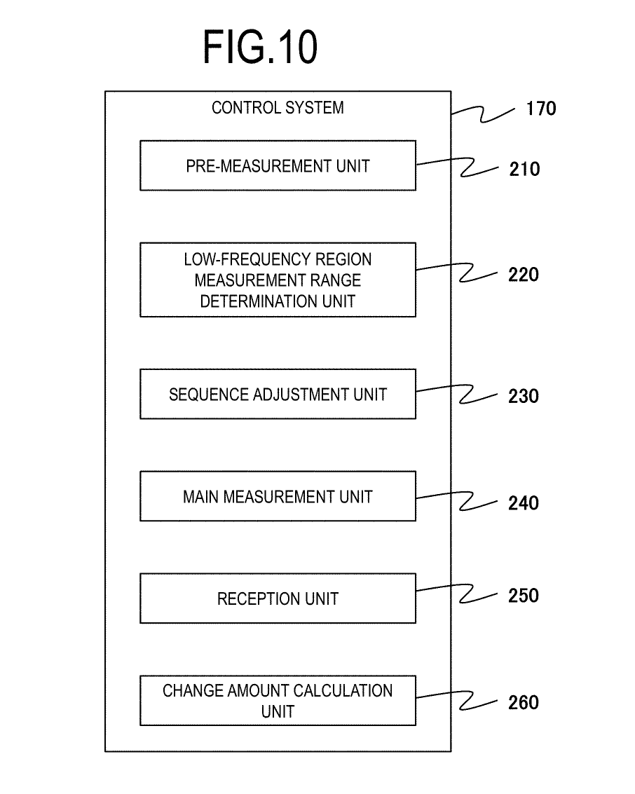

In order to realize this, an MRI apparatus of the present embodiment fundamentally has the same configuration as that of the MRI apparatus 100 of the first embodiment. However, in order to realize the above-described function, the control system 170 of the present embodiment includes, as illustrated in FIG. 10, a reception unit 250 and a change amount calculation unit 260 in addition to the configuration in the first embodiment. Hereinafter, the present embodiment will be described focusing on configurations which are different from the first embodiment.

The change amount calculation unit 260 calculates a change amount to be changed so that measurement time in an imaging sequence (adjusted sequence) after being adjusted is the same as measurement time in an imaging sequence (initial sequence) before being adjusted with respect to predefined measurement parameters to be changed other than the measurement time. Hereinafter, measurement time in an adjusted sequence being the same as measurement time in an initial sequence will be referred to as measurement time being maintained.

The measurement time in the adjusted sequence is calculated by the low-frequency region measurement range determination unit 220 on the basis of determined k space characteristic information. The change amount is calculated as an amount of changing measurement time by a difference between the measurement time in the initial sequence and the measurement time in the adjusted sequence with respect to a designated measurement parameter. For example, in TR, a difference is calculated as a change amount without being changed.

The reception unit 250 receives selection regarding whether either measurement time in an adjusted imaging sequence (adjusted sequence) or predefined measurement parameters other than the measurement time are fixed. In the present embodiment, the reception unit 250 displays an instruction reception screen on the display device and receives an instruction from a user. The reception unit 250 presents a change amount calculated by the change amount calculation unit and measurement time in an imaging sequence (adjusted sequence) adjusted by the sequence adjustment unit to the user on the instruction reception screen, and receives selection.

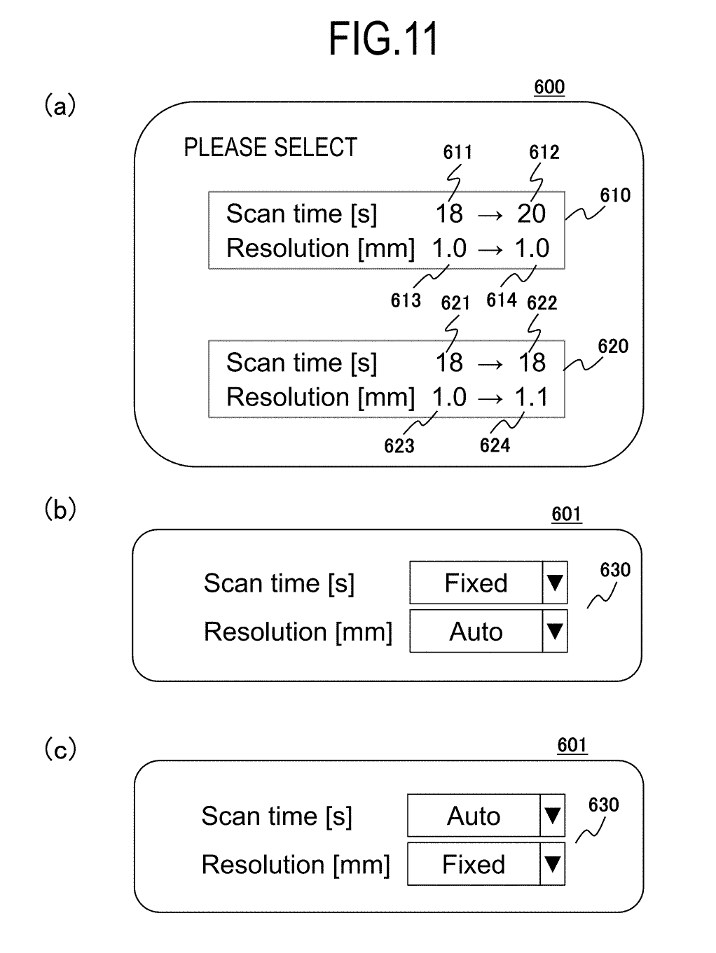

FIG. 11(a) illustrates an example of an instruction reception screen 600. As illustrated in this figure, the instruction reception screen 600 displays not only a change of adjusted measurement time but also changes of other predetermined measurement parameters in a case where the measurement time is not changed. Here, as an example, a description will be made of a case where a resolution is used as any one of other measurement parameters.

The instruction reception screen 600 is a screen for receiving selection regarding whether measurement time is changed or a spatial resolution is changed. As illustrated in this figure, the instruction reception screen includes a first display column 610 which displays measurement time in a case where the adjusted sequence is performed, and a second display column 620 which displays a resolution in a case where measurement time is maintained in the adjusted sequence.

In the present embodiment, as illustrated in this figure, the first display column 610 displays measurement time (scan time) 611 in the initial sequence and measurement time (scan time) 612 in the adjusted sequence, and presents a change thereof to the user. In this case, for comparison with the second display column 620, a resolution (resolution) 613 in the initial sequence and a resolution (resolution) 614 in the adjusted sequence are also displayed so that the resolution is not changed. Regarding the resolution, an example is shown in which, a value obtained by normalizing a resolution in the initial sequence into 1 is displayed.

Similarly, the second display column 620 displays measurement time (scan time) 621 in the initial sequence and measurement time (scan time) 622 in the adjusted sequence. The second display column 620 performs display in a case where measurement time in the adjusted sequence is maintained, and thus displays the same time. A resolution (resolution) 623 in the initial sequence and a resolution (resolution) 624 in the adjusted sequence are displayed. The resolution 624 in the adjusted sequence displays a calculation result in the change amount calculation unit 260.

The reception unit 250 receives selection from the user via the first display column 610 or the second display column 620.

The sequence adjustment unit 230 of the present embodiment reflects the received result in the adjusted sequence, and finally adjusts the adjusted sequence to a sequence used for imaging. In other words, in a case where the measurement time is selected, a measurement parameter in the imaging sequence (adjusted sequence) is adjusted by a change amount calculated by the change amount calculation unit.

Processes in the low-frequency region measurement range determination unit 220, the pre-measurement unit 210, and the main measurement unit of the present embodiment are the same as those in the first embodiment. For the sequence adjustment unit 230, processes until an adjusted sequence is generated are the same as those in the first embodiment.

As described above, the MRI apparatus 100 of the present embodiment includes the pre-measurement unit 210, the low-frequency region measurement range determination unit 220, the sequence adjustment unit 230, and the main measurement unit 240 in the same manner as in the first embodiment. The MRI apparatus 100 of the present embodiment further includes the reception unit 250 which receives selection regarding whether measurement time in an imaging sequence after being adjusted is fixed or a predefined measurement parameter other than the measurement time is fixed; and the change amount calculation unit 260 which calculates a change amount to be changed so that the measurement time in the imaging sequence after being adjusted is the same as measurement time in an imaging sequence before being adjusted with respect to the measurement parameter, in which, in a case where the measurement time is selected, the sequence adjustment unit 230 further adjusts the measurement parameter in the imaging sequence by the change amount.

The reception unit 250 may present the change amount and the measurement time in the imaging sequence adjusted by the sequence adjustment unit 230 to a user, so as to receive the selection.

According to the present embodiment, it is possible to achieve the same effect as that in the first embodiment. Since a user can select whether measurement time is permitted to increase in an adjusted sequence, or measurement time is maintained by adjusting other parameters, it is possible to realize desired measurement within desired measurement time.

### Modification Example of UI

In the above-described embodiment, measurement time in an adjusted sequence, and a change amount of a measurement parameter in a case where the measurement time is maintained are displayed on the instruction reception screen 600, and selection is received from a user. However, information displayed on the instruction reception screen 600 is not limited thereto.

For example, whether or not a variable measurement parameter also including measurement time is changed may be received without displaying information such as measurement time in an initial sequence and an adjusted sequence on the instruction reception screen 600. The instruction reception screen 600 may be provided with a region for receiving an instruction for priority of adjustment with respect to each variable measurement parameter. In this case, if measurement time is increased, the measurement time or other measurement parameters are changed by taking into consideration the priority.

FIGS. 11(b) and 11(c) illustrate examples of an instruction reception screen 601 in a case where an instruction for the priority is received. Also here, as an example, a description will be made of a case where a resolution (resolution) is used as any one of measurement parameters other than measurement time.

In this case, the instruction reception screen 601 has an Instruction column 630 for receiving an instruction for preferentially adjusting one of measurement time and a resolution.

FIG. 11(b) illustrates an example of a case of receiving an instruction for measurement time being fixed (maintained; fixed), and a resolution being automatically changed (Auto) according thereto. In other words, in this example, an instruction for preferentially adjusting a resolution is received.

FIG. 11(c) illustrates an example of a case of receiving an instruction for measurement time being automatically changed (Auto), and a resolution being maintained (fixed). In other words, in this example, an instruction for preferentially adjusting measurement time is received.

The priority is set for measurement time and a spatial resolution here, but the priority may be set for other parameters (for example, TR).

In a case where there are many parameters f or which Auto is set, and thus a parameter to be changed is not uniquely defined, parameters may be changed by displaying an option on the instruction reception screen 601, and allowing a user to select any option.

Instead of displaying and selecting whether a parameter is maintained or changed, an instruction may be received by displaying a change within a predetermined range.

In other words, the reception unit 250 receives selection regarding whether either measurement time in an imaging sequence after being adjusted (adjusted sequence) or predefined measurement parameters other than the measurement time are changed only within a predefined range. The change amount calculation unit 260 calculates a change amount to be changed so that a difference between measurement time in an imaging sequence (adjusted sequence) after being adjusted and measurement time in an imaging sequence before being adjusted (initial sequence) is included in the range with respect to the measurement parameters.

In this case, if the measurement time is selected, the sequence adjustment unit 230 also adjusts the measurement parameters according to a calculation result in the change amount calculation unit 260 in the imaging sequence (adjusted sequence).

In this case, for example, the range may be presented along with fixation (Fixed) on the instruction reception screen 601 illustrated in FIG. 11(b) or 11(c). There may be a configuration in which the range can be set by a user.

The priority is configured to be selected, and thus it is possible to reduce time and effort of a user. A fixed range is set, and thus it is possible to realize more detailed adjustment.

### Third Embodiment

Next, a third embodiment of the present invention will be described. In the first embodiment, the pre-measurement unit 210 measures all measurement points in a predefined search range during pre-measurement. On the other hand, in the present embodiment, a process is performed at a high speed by reducing the number of measurement points in pre-measurement.

An MRI apparatus of the present embodiment fundamentally has the same configuration as that of the MRI apparatus 100 of the first embodiment. However, as described above, in order to reduce the number of measurement points and to estimate a deficient amount, processes in the pre-measurement unit 210 and the low-frequency region measurement range determination unit 220 are different. Hereinafter, the present embodiment will be described focusing on configurations which are different from the first embodiment.

In the present embodiment, whenever the pre-measurement unit 210 acquires k space data, the low-frequency region measurement range determination unit 220 estimates k space characteristic information (a k space reference position and a k space low-frequency region measurement width), estimates a k space low-frequency region data measurement range by using the k space characteristic information, and finishes pre-measurement at the time of convergence. In a case where convergence does not occur even if measurement is performed in the entire k space search range, pre-measurement is finished at the time of completing measurement in the entire k space search range. Hereinafter, pieces of information to be estimated will be respectively referred to as estimation k space characteristic information, an estimation k space reference position, an estimation k space low-frequency region measurement width, and an estimation k space low-frequency region data measurement range.

The pre-measurement unit 210 measures k space data in predefined order, and disposes the k space data in the k space whenever the k space data is measured.

The low-frequency region measurement range determination unit 220 estimates non-measured k space data whenever the pre-measurement unit 210 acquires k space data, and estimates estimation k space characteristic information and estimation k space low-frequency region data measurement range by using the estimated result, and, in a case where a difference from an estimation k space low-frequency region data measurement range which is previously estimated falls within a predefined range, the low-frequency region measurement range determination unit sets the latest estimation k space characteristic information at that time as k space characteristic information.

In other words, the low-frequency region measurement range determination unit 220 estimates a signal (search data) which is not measured when the pre-measurement unit 210 disposes k space data in the k space, estimates estimation k space characteristic information, and estimates an estimation k space low-frequency region data measurement range on the basis of the estimation k space characteristic information (an estimation k space reference position and an estimation k space low-frequency region measurement width) which has been estimated.

The low-frequency region measurement range determination unit 220 determines propriety of the estimated result. The propriety is determined by calculating a difference from an estimation k space low-frequency region data measurement range which is previously estimated, and on the basis of whether or not the difference is included in a predefined threshold value. If the difference is included in the threshold value, convergence is determined, the pre-measurement unit 210 finishes measurement, and the latest estimation k space characteristic information at that time is output as a determination result.

The measurement is also finished in a case where the pre-measurement unit 210 completes measurement in the entire search range while convergence does not occur. In this case, the low-frequency region measurement range determination unit 220 outputs k space characteristic information which is determined by using all pieces of k space data obtained at that time, as a determination result.

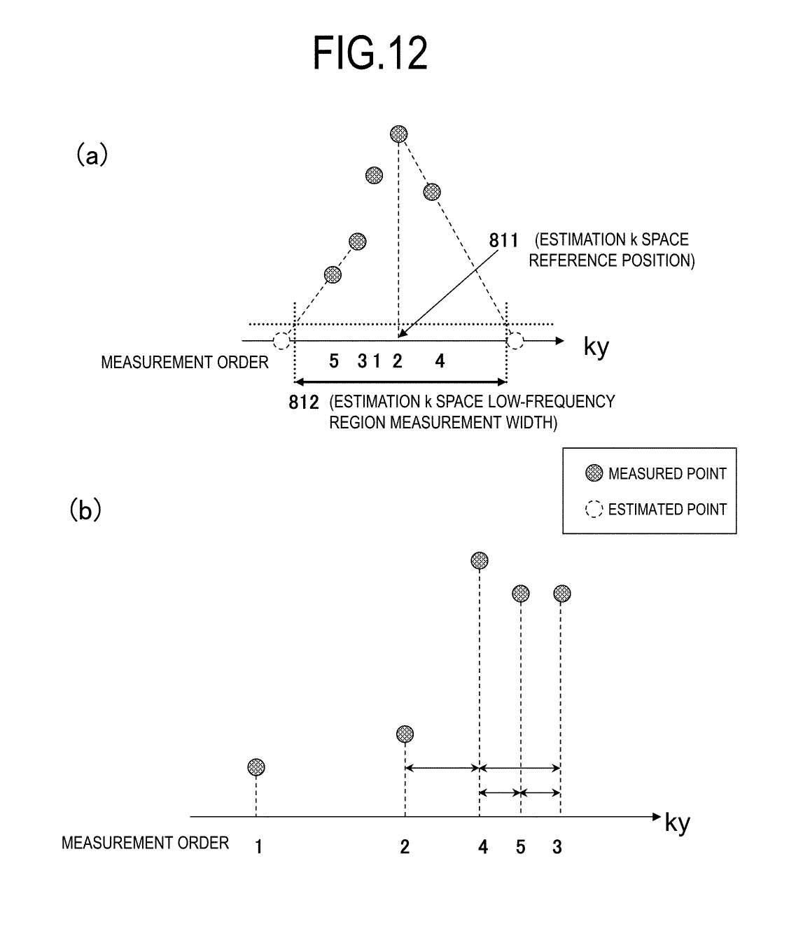

With reference to FIG. 12(a), details of estimation in the low-frequency region measurement range determination unit 220.

Here, as an example, a description will be made of an example of a case where the pre-measurement unit 210 changes a phase encode amount from 0 so that an absolute value thereof monotonously increases, and thus acquires k space data. In other words, the pre-measurement unit 210 changes a phase encode amount to a greater absolute value in the order of 0, +1, −1, +2, and −2, and thus acquires k space data.

As illustrated in this figure, the low-frequency region measurement range determination unit 220 estimates another measurement point (estimated data) by using an estimated measurement point (measured data). The estimation is performed through extrapolation from a measured point (estimated data). In FIG. 12(a), primary extrapolation from outer two points among measured points is used, but any number of points or any order may be used. An estimated result is held in a memory.

The low-frequency region measurement range determination unit 220 determines estimation k space characteristic information (an estimation k space reference position and an estimation k space low-frequency region measurement width) according to each method in the first embodiment by using the measured data and the estimated data.

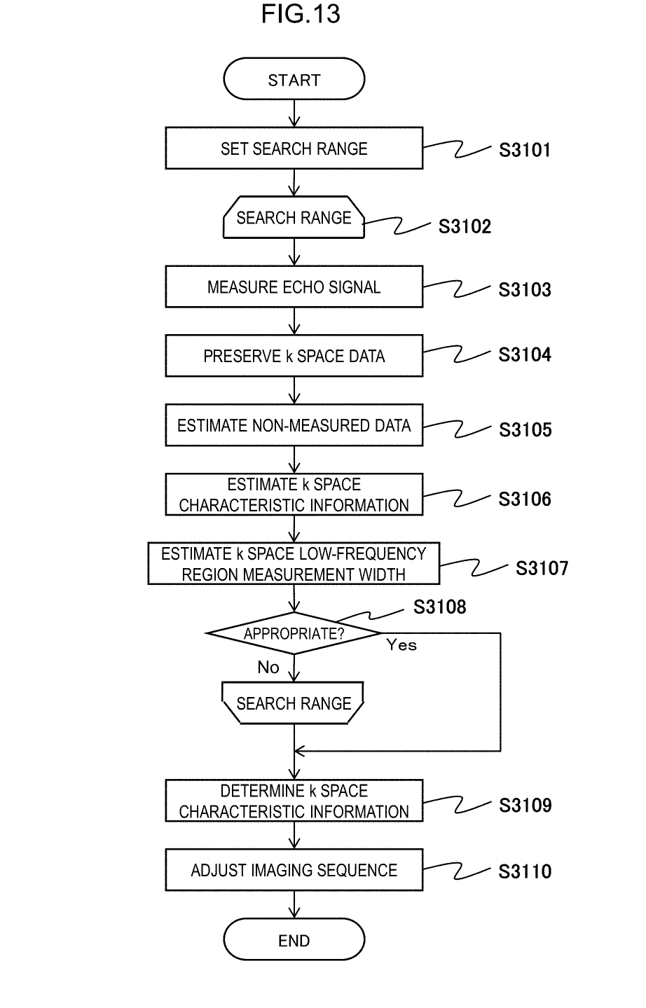

A description will be made of a flow of a k space characteristic information determination process and an imaging sequence adjustment process in the present embodiment. FIG. 13 illustrates a processing flow of the present process. Also in the present embodiment, the present process is performed right after each scanning is started prior to main measurement.

The pre-measurement unit 210 sets a search range for pre-measurement in order to determine k space characteristic information (step S3101), and starts pre-measurement in the search range (step S3102).

In the pre-measurement, the pre-measurement unit 210 measures an echo signal (step S3103), and preserves k space data whenever the k space data is acquired (step S3104).

If the pre-measurement unit 210 preserves the k space data, the low-frequency region measurement range determination unit 220 estimates non-measured data (step S3105), estimates k space characteristic information (estimation k space characteristic information) by using measured data and non-measured data (step S3106), and based on the estimated result, estimates a k space low-frequency region data measurement range (estimation k space low-frequency region data measurement range) (step S3107). The estimation k space low-frequency region data measurement range which has been estimated is preserved in the memory.

As described above, the low-frequency region measurement range determination unit 220 obtains a difference from an estimation k space low-frequency region data measurement range which is obtained through the previous measurement, so as to determine propriety (step S3108).

In a case where impropriety is determined, and the entire search range is not searched, the low-frequency region measurement range determination unit 220 updates the estimation k space low-frequency region data measurement range which is obtained through the previous measurement and is preserved in the memory to the latest estimation k space low-frequency region data measurement range, and returns to step S3103 so as to repeatedly perform the processes.

On the other hand, in a case where propriety is determined, or the entire search range has been searched, the low-frequency region measurement range determination unit 220 outputs the latest estimation k space characteristic information at that time as a determination result (step S3109).

The sequence adjustment unit 230 adjusts an imaging sequence on the basis of the k space characteristic information (the k space reference position and the k space low-frequency region measurement width) determined by the low-frequency region measurement range determination unit 220 (step S3110).

As described above, the MRI apparatus 100 of the present embodiment includes the pre-measurement unit 210, the low-frequency region measurement range determination unit 220, the sequence adjustment unit 230, and the main measurement unit 240 in the same manner as in the first embodiment. The low-frequency region measurement range determination unit 220 estimates k space data which is not measured whenever the pre-measurement unit 210 acquires the k space data, and estimates estimation k space characteristic information and estimation k space low-frequency region data measurement range by using the estimated result, and, in a case where a difference from an estimation k space low-frequency region data measurement range which is previously estimated falls within a predefined range, the low-frequency region measurement range determination unit sets the latest, estimation k space characteristic information at that time as the k space characteristic information.

As mentioned above, according to the present embodiment, it is possible to achieve the same effect as that in the first embodiment. Since the number of measurement points can be reduced when k space characteristic information is obtained, the whole measurement time is reduced according thereto. Therefore, it is possible to achieve the same effect as in the first embodiment at a higher speed.

### Modification Example of Measurement Order

The measurement order of the k space in the pre-measurement unit 210 is not limited to the above description. For example, the measurement order as illustrated in FIG. 12(b) may be employed.

In this measurement order, first, the respective points corresponding to the measurement order of 1, 2, and 3 are measured. These points are two points substantially located at both ends of the search range and one point located at the center. Next, a signal average value corresponding to the measurement order of 1 and 2 and a signal average value corresponding to the measurement order of 2 and 3 are obtained, and the midpoint (here, the midpoint in the measurement order of 2 and 3) of a greater average value is measured in the measurement order of 4. Similarly, measurement is performed in the measurement order of 5 on the basis of a signal average value corresponding to the measurement order of 2 and 4 and a signal average value corresponding to the measurement order of 4 and 3. This is repeatedly performed.

By employing such measurement order, it is possible to estimate an estimation k space reference position with a small number of measurement and thus to estimate estimation k space characteristic information. The number of times of repetition in this case may be determined according to convergence of a k space reference position, and may be obtained on the basis of a search range.

The present embodiment is applicable to the first embodiment and the respective modification examples thereof. A UI may be provided as in the second embodiment and modification examples thereof.

### Fourth Embodiment

A fourth embodiment of the present invention will be described. In the present embodiment, k space search data which is obtained through pre-measurement is used not only for determining k space characteristic information (a k space reference position and a k space low-frequency region measurement width) but also for setting an optimal reception gain.

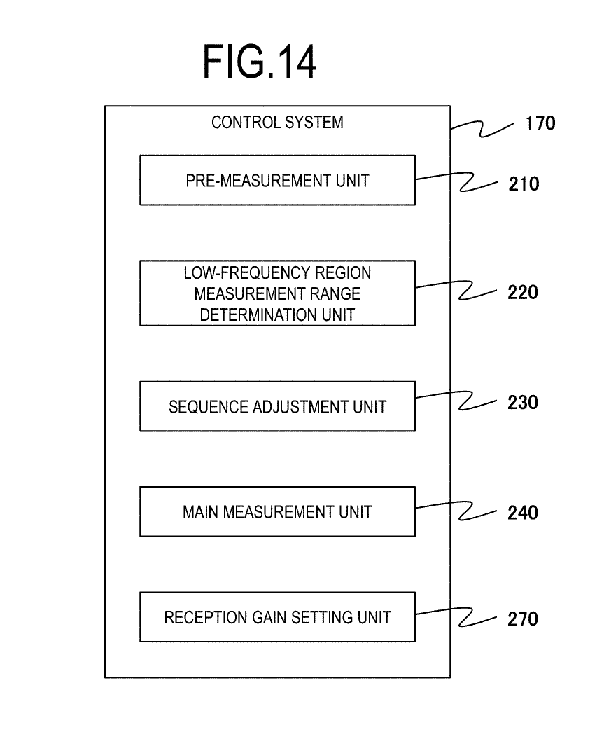

An MRI apparatus of the present embodiment fundamentally has the same configuration as that of the MRI apparatus 100 of the first embodiment. However, as illustrated in FIG. 14, the control system 170 of the present embodiment further includes a reception gain setting unit 270 which determines a reception gain corresponding to a position in the k space on the basis of k space data obtained through pre-measurement. Hereinafter, the present embodiment will be described focusing on configurations which are different from the first embodiment.

The reception gain setting unit 270 calculates the maximum reception gain which is applicable to each position in a k space low-frequency region according to a shape of k space low-frequency region data which is calculated when the low-frequency region measurement range determination unit 220 determines k space characteristic information (a k space reference position and a k space low-frequency region measurement width).

Specifically, an amplification ratio of an echo signal from a position in an amplifier of the signal reception processing unit 162 is determined according to the maximum value of the signal intensity corresponding to the position in the k space low-frequency region. The amplification ratio is determined to maximally use a dynamic range of an A/D converter disposed in the subsequent stage of the amplifier in the signal reception processing unit 162. An instruction for a determination result is given to the signal reception processing unit 162.

The signal reception processing unit 162 amplifies each echo signal received by the signal reception coil 161 in response to an instruction during main measurement. After the echo signals are amplified, a difference in signal amplification due to a difference between reception gains is normalized, and then the echo signals are output. Consequently, the control system 170 can perform a reconstruction process in the same manner as in typical image data.

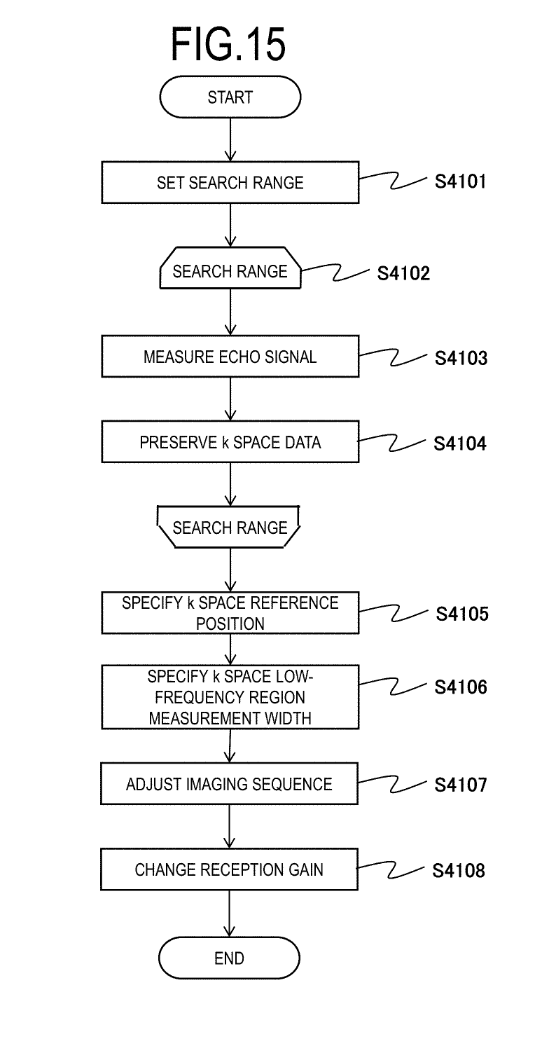

A description will be made of a flow of a k space low-frequency region data measurement range determination process, an imaging sequence adjustment process, and a reception gain setting process in the present embodiment. FIG. 15 illustrates a processing flow of the present process. The present process is performed right after an instruction for starting each scanning is given prior to main measurement.

Processes in steps S4101 to S4107 are the same as the processes in steps S1101 to S1107 in the first embodiment, and thus detailed description thereof will be omitted here.

If adjustment of the imaging sequence is completed, the reception gain setting unit 270 determines a reception gain corresponding to a position in the k space on the basis of the shape of the k space low-frequency region data obtained in order to calculate k space characteristic information, and notifies the signal reception processing unit 162 thereof so as to change a reception gain (step S4108), and finishes the process.

As described above, the MRI apparatus 100 of the present embodiment includes the pre-measurement unit 210, the low-frequency region measurement range determination unit 220, the sequence adjustment unit 230, and the main measurement unit 240 in the same manner as in the first embodiment. The MRI apparatus 100 of the present embodiment further includes the signal reception processing unit 162 which amplifies an echo signal collected by the signal reception coil so as to obtain the k space data, and the reception gain setting unit 270 which determines a reception gain corresponding to a position in the k space on the basis of the k space data obtained through the pre-measurement.

Generally, a reception gain takes a single value for each measurement. However, according to the present embodiment, it is possible to calculate the maximum reception gain according to a position in the k space in accordance with k space characteristics which are specified on the basis of k space search data. Consequently, the amplifier and the A/D converter of the signal reception processing unit 162 are adjusted.

Signals are received at the optimum reception gain, and thus it is possible to perform signal reception maximally using a dynamic range of the A/D converter. Consequently, it is possible to prevent a reduction in an SNR due to mixture of system noise.

The respective modification examples of the first embodiment, and the UI of the second embodiment and the modification examples thereof are also applicable to the present embodiment. As a k space low-frequency region data shape, a k space low-frequency region data shape estimated according to the method of the third embodiment may be used.

In the above-described respective embodiments, two-dimensional measurement has been described as an example, but the above-described respective embodiments are also applicable to a case of three-dimensional measurement. In a case of three-dimensional measurement, a phase encode amount and the number of steps, and a slice encode amount and the number of steps, used for the k space low-frequency region, are determined, and an imaging sequence is adjusted.

### REFERENCE SIGNS LIST

100 MRI Apparatus, 101 Object, 120 Static Magnetic Field Generation System, 130 Gradient Magnetic Field Generation System, 131 Gradient Magnetic Field Coil, 132 Gradient Magnetic Field Power Source, 150 Signal Transmission System, 151 High Frequency Coil (Signal Transmission Coil), 152 Signal Transmission Processing Unit, 160 Signal Reception Processing Unit, 161 High Frequency Coil (Signal Reception Coil), 162 Signal Reception Processing Unit, 170 Control System, 171 CPU, 172 Storage Device, 173 Display Device, 174 Input Device, 210 Pre-Measurement Unit, 220 Low-Frequency Region Measurement Range Determination Unit, 230 Sequence Adjustment Unit, 240 Main Measurement Unit, 250 Reception Unit, 260 Change Amount Calculation Unit, 270 Reception Gain Setting Unit, 301 Shape of k Space Search Data of Channel 1, 302 Shape of k Space Search Data of Channel 2, 303 Shape of Addition k Space Search Data, 310 k Space Reference Position, 311 Peak Position of Channel 1, 312 Peak Position of Channel 2, 313 k Space Reference Position, 410, 420, and 430 k Space Low-Frequency Region Measurement Width, 440a k Space Low-Frequency Region Measurement Width After OR Combination, 440b k Space Low-Frequency Region Measurement Width After AND Combination, 440c k Space Low-Frequency Region Measurement Width After Centroid Combination, 441 k Space Low-Frequency Region Measurement Width of Channel 1, 442 k Space Low-Frequency Region Measurement Width of Channel 2, 501 Initial Phase Encode Gradient Magnetic Field, 502, 503, and 504 Phase Encode Gradient Magnetic Field After Adjustment, 511 Initial Sampling Density, 512, 513, and 514 Sampling Density After Adjustment, 600 and 601 Instruction Reception Screen, 610 First Display Column, 611 Measurement Time in Initial Sequence, 612 Measurement Time in Adjusted Sequence, 613 Resolution in Initial Sequence, 614 Resolution in Adjusted Sequence, 620 Second Display Column, 621 Measurement Time in Initial Sequence, 622 Measurement Time in Adjusted Sequence, 623 Resolution in Initial Sequence, 624 Resolution in Adjusted Sequence, 630 Instruction Column

1. A magnetic resonance imaging apparatus comprising:
a pre-measurement unit that measures k space data in a k space low-frequency region which is a predefined low-frequency region range of a k space by using the same imaging sequence as an imaging sequence in main measurement performed for acquiring an image;
a low-frequency region measurement range determination unit, that obtains k space characteristic information for specifying a k space low-frequency region data measurement range in which k space low-frequency region data can be measured by using the k space data collected by the pre-measurement unit;
a sequence adjustment unit that adjusts the imaging sequence so that k space data in the k space low-frequency region data measurement range is measured as the k space low-frequency region data; and
a main measurement unit that performs the main measurement by using an imaging sequence adjusted by the sequence adjustment unit.

2. The magnetic resonance imaging apparatus according to claim 1,
wherein the k space characteristic information includes a k space reference position which is a position where the signal intensity of the k space data is the maximum; and
wherein the sequence adjustment unit adjusts the imaging sequence so that the k space low-frequency region data is measured from a range centering on the k space reference position.

3. The magnetic resonance imaging apparatus according to claim 1,
wherein the k space characteristic information includes a k space low-frequency region measurement width defined according to the signal intensity of the k space data, and
wherein the sequence adjustment unit adjusts the imaging sequence so that the k space low-frequency region data is measured from a range of the k space low-frequency region measurement width.

4. The magnetic resonance imaging apparatus according to claim 2, further comprising:
a multi-channel signal reception coil,
wherein the low-frequency region measurement range determination unit sets a position where the signal intensity of combined data obtained by combining pieces of the k space data received in respective channels of the signal reception coil with each other is the maximum, as the k space reference position.

5. The magnetic resonance imaging apparatus according to claim 3, further comprising:
a multi-channel signal reception coil,
wherein the low-frequency region measurement range determination unit sets a range which includes a k space reference position which is a position where the signal intensity of the k space data is the maximum and in which combined data obtained by combining pieces of the k space data received in respective channels of the signal reception coil with each other satisfies a predefined condition, as the k space low-frequency region measurement width.

6. The magnetic resonance imaging apparatus according to claim 1, further comprising:
a reception unit that receives selection regarding whether either one of measurement time in an imaging sequence after the adjustment and a predefined measurement parameter other than the measurement time is fixed; and
a change amount calculation unit that calculates a change amount to be changed so that measurement time in the adjusted imaging sequence is the same as measurement time in an imaging sequence before being adjusted, with respect to the measurement parameter,
wherein, in a case where the measurement time is selected, the sequence adjustment unit further adjusts the measurement parameter in the imaging sequence by the change amount.

7. The magnetic resonance imaging apparatus according to claim 6,
wherein the reception unit presents the change amount and the measurement time in the imaging sequence adjusted by the sequence adjustment unit to a user, and receives the selection.

8. The magnetic resonance imaging apparatus according to claim 1,
wherein the low-frequency region measurement range determination unit estimates non-measured k space data whenever the pre-measurement unit acquires the k space data, and estimates estimation k space characteristic information and estimation k space low-frequency region data measurement range by using the estimated result, and, in a case where a difference from estimation k space low-frequency region data measurement range which is previously estimated falls within a predefined range, the low-frequency region measurement range determination unit sets the latest estimation k space characteristic information at that time as the k space characteristic information.

9. The magnetic resonance imaging apparatus according to claim 1, further comprising:
a signal reception processing unit that amplifies an echo signal collected by the signal reception coil so as to obtain the k space data; and
a reception gain setting unit that determines a reception gain corresponding to a position in a k space on the basis of the k space data obtained by the pre-measurement unit.

10. The magnetic resonance imaging apparatus according to claim 2, further comprising:
a multi-channel signal reception coil,
wherein the low-frequency region measurement range determination unit specifies a position where the signal intensity is the maximum with respect to each piece of the k space data received in the respective channels of the signal reception coil, and sets a centroid position of each specified result as the k space reference position.

11. The magnetic resonance imaging apparatus according to claim 3, further comprising:
a multi-channel signal reception coil,
wherein the low-frequency region measurement range determination unit specifies a region which includes a k space reference position which is a position where the signal intensity of the k space data is the maximum and in which pieces of the k space data received in respective channels of the signal reception coil satisfy a predefined condition, and obtains the k space low-frequency region measurement width by combining specified results with each other.

12. The magnetic resonance imaging apparatus according to claim 1, further comprising:
a reception unit that receives selection regarding whether either one of measurement time in an imaging sequence after the adjustment and a predefined measurement parameter other than the measurement time is changed only within a predefined range; and
a change amount calculation unit that calculates a change amount to be changed so that a difference between measurement time in the adjusted imaging sequence and measurement time in an imaging sequence before being adjusted falls within the range, with respect to the measurement parameter,
wherein, in a case where the measurement time is selected in the reception unit, the sequence adjustment unit also adjusts the measurement parameter according to a calculation result in the change amount calculation unit in the imaging sequence.

13. A magnetic resonance imaging method comprising:
collecting k space data in a predefined range of a k space low-frequency region by using the same imaging sequence as an imaging sequence in main measurement performed for acquiring an image;
specifying a k space low-frequency region data measurement range for measuring k space low-frequency region data by using the collected k space data;
adjusting the imaging sequence so that the k space low-frequency region data is measured from the k space low-frequency region data measurement range; and
performing the main measurement by using the adjusted imaging sequence.
